(19) United States
(12) Patent Application Publication
(10) Pub. No.: US 2012/0059120 A1
(43) Pub. Date: Mar. 8, 2012

(54) Carbon Nanotube Grafted with Low-Molecular Weight Polyaniline and Dispersion Thereof

(76) Inventors: Tatsuhiro TAKAHASHI, Yonezawa-shi (JP); Noriyuki KURAMOTO, Yonezawa-shi (JP); Koichiro YONETAKE, Yonezawa-shi (JP); Osamu HABA, Yonezawa-shi (JP); Hiroshi AWANO, Yonezawa-shi (JP); Hiroki ARAI, Yonezawa-shi (JP); Teruya GOTO, Yonezawa-shi (JP); Yushi YAMAGUCHI, Yonezawa-shi (JP); Naoya NISHIMURA, Funabashi-shi (JP); Masahiro HIDA, Funabashi-shi (JP); Masaaki OZAWA, Funabashi-shi (JP); Mitsunobu MATSUMURA, Funabashi-shi (JP)

(21) Appl. No.: 13/293,833

(22) Filed: Nov. 10, 2011

Related U.S. Application Data
(62) Division of application No. 12/270,687, filed on Nov. 13, 2008.

(30) Foreign Application Priority Data
May 14, 2008 (JP) 2008-126695

(51) Int. Cl. C07C 211/54; B82Y 40/00

(52) U.S. Cl. 524/612; 977/847

(57) ABSTRACT

Chemically modified carbon nanotubes composed of carbon nanotubes (such as multiwall carbon nanotubes) having carboxyl groups on the surface thereof and polymeric aniline (such as 3- to 300-meric aniline) bonding thereto through the amide linkage. The chemically modified carbon nanotubes exhibit good affinity with organic solvents and readily disperse into organic solvents.

This application is a Divisional of U.S. application Ser. No. 12/270,687, filed on Nov. 13, 2008, which claims priority from Japanese Patent Application No. 2008-126695, filed on May 14, 2008, the entire contents of which are herein incorporated by reference into the present application and for which priority is claimed under 35 U.S.C. §120.

### TECHNICAL FIELD

The present invention relates to carbon nanotubes whose surface is chemically modified and, more particularly, to chemically modified carbon nanotubes wherein the modifier is oligo- or polyaniline.

### BACKGROUND ART

Carbon nanotubes (CNT for short hereinafter) are regarded as one of the useful materials in the field of nanotechnology.

Their applications are divided into two classes. In the first one, CNT is used alone as a transistor or a probe for a microscope. In the second one, CNT is used as an electron emitting electrode, a fuel cell electrode, or a conductive composite material containing CNTs dispersed therein. These applications employ a large number of CNTs in the form of bulk.

For CNTs to be used individually, CNTs are added to a solvent and irradiated with ultrasonic waves for dispersion and dispersed CNTs are collected by electrophoresis.

In the case of use in the form of bulk for conductive composite material, CNTs are uniformly incorporated into the matrix such as polymer.

Unfortunately, CNTs are usually difficult to disperse, and ordinary means for dispersion does not give rise to a composite material containing uniformly dispersed CNTs. To achieve good dispersion, there have been proposed several methods for surface modification of CNTs.

One of such methods is treatment of CNTs with an aqueous solution containing a surfactant such as sodium dodecylsulfonate (see Patent Document 1: JP-A 6-228824). This method suffers the disadvantage of contaminating the surface of CNTs with a non-conductive organic material which deteriorates conductivity.

Another known method is by coating the surface of CNTs with a polymer having the coil structure. Specifically, this method consists of adding CNTs into a solvent containing poly-m-phenylenevinylene-co-dioctoxy-p-phenylenevinylene to precipitate a CNT composite material, followed by separation and purification (Patent Document 2: JP-A 2000-44216). The disadvantage of this method is that the polymer is incomplete in the conjugated system, and this impairs the conductivity of CNTs.

Other methods include the surface modification of CNTs with carboxyl groups (Patent Document 3: U.S. Pat. No. 6,368,569), with amino groups (Patent Documents 4 and 5: U.S. Pat. No. 6,187,823 and U.S. Pat. No. 6,331,262), or with guanidine groups (Patent Document 6: JP-A 2006-206568). They are poor in dispersion.

For development of CNTs with improved conductivity, there have been proposed several methods for hybridization of CNT with a polymer of every kind.

Among the known products of hybridization is a CNT-polyaniline hybrid composite (Non-Patent Document 1: European Polymer Journal, 38. 2002, p. 2497-2501). However, this composite is also poor in dispersion.

Patent Document 1: JP-A 6-228824

Patent Document 2: JP-A 2000-44216

Patent Document 3: U.S. Pat. No. 6,368,569

Patent Document 4: U.S. Pat. No. 6,187,823

Patent Document 5: U.S. Pat. No. 6,331,262

Patent Document 6: JP-A 2006-206568

Non-Patent Document 1: European Polymer Journal, 38. 2002, p. 2497-2501

### DISCLOSURE OF THE INVENTION

### Problems To Be Solved By the Invention

The present invention was completed in view of the foregoing. It is an object of the present invention to provide chemically modified carbon nanotubes characterized by good affinity with organic solvents and good dispersibility in organic solvents.

### Means For Solving the Problems

After their intensive researches to achieve the foregoing object, the present inventors found that carbon nanotubes having carboxyl groups introduced into their surface exhibit good affinity with organic solvents and good dispersibility in organic solvents when they are chemically modified by grafting with polymeric aniline through the amide linkage. They also found that the chemically modified carbon nanotubes are readily dispersible into an organic solvent and the resulting dispersion can be made into a thin film in which the carbon nanotubes are uniformly distributed to form a network structure. These findings led to the present invention.

The gist of the present invention resides in:
(1) Chemically modified carbon nanotubes which comprise carbon nanotubes having carboxyl groups on the surface thereof and polymeric aniline bonding thereto through the amide linkage.
(2) The chemically modified carbon nanotubes according to (1) above, wherein said carbon nanotubes are multiwall carbon nanotubes.
(3) The chemically modified carbon nanotubes according to (1) or (2) above, wherein said carbon nanotubes contain said carboxyl groups in an amount of 0.1 to 1 mmol/g.
(4) The chemically modified carbon nanotubes according to any of (1) to (3) above, wherein said polymeric aniline is 3- to 300-meric aniline.
(5) A composition of the chemically modified carbon nanotubes according to any of (1) to (4) above which is dispersed in an organic solvent.
(6) A thin film obtained from the composition according to (5) above.

### Effects of the Invention

The carbon nanotubes according to the present invention, having their surface modified with polymeric aniline, exhibit good affinity with organic solvents and readily disperse into organic solvents.

The carbon nanotubes in the form of fluid dispersion gives rise to a thin film in which they uniformly disperse to form a network structure.

The thin film containing the carbon nanotubes according to the present invention will find use as a semiconducting material and a conducting material.

### BEST MODE FOR CARRYING OUT THE INVENTION

The following is a detailed description of the present invention.

The chemically modified carbon nanotubes according to the present invention are composed of carbon nanotubes having carboxyl groups on the surface thereof and polymeric aniline bonding thereto through the amide linkage.

Ordinary carbon nanotubes (CNT) are produced by arc discharging method, chemical vapor deposition method, or laser ablation method. The CNT used in the present invention may be produced by any of them. CNT exists in three forms—single-wall CNT (SWCNT) consisting of one graphene sheet in a cylindrical shape, double-wall CNT (DWCNT) consisting of two graphene sheets in a coaxially wound shape, and multiwall CNT (MWCNT) consisting of more than two graphene sheets in a coaxially wound shape. In the present invention, SWCNT, DWCNT, and MWCNT may be used alone or in combination with one another.

According to the present invention, no restrictions are specifically imposed on the amount of carboxyl groups on the surface of CNT. However, an adequate amount is preferably 0.1 to 1 mmol/g, more preferably 0.3 to 0.7 mmol/g, which is necessary for the CNT to exhibit good dispersibility by grafting with a certain amount of polymeric aniline.

Introduction of carboxyl groups into the surface of CNT may be accomplished by the method disclosed by Goh, H. W., Goh, S. H., Xu, G. Q., Pramoda, K. P., Zhang, W. D. “Crystallization and dynamic mechanical behavior of double-C-60-end-capped poly(ethylene oxide)/multi-walled carbon nanotube composites” Chem. Phys. Lett. 379 236-241 (2003).

The polymeric aniline may be produced by any method without specific restrictions, such as the one disclosed by W. J. Zhang, J. Feng, A. G. MacDiarmid, and A. J. Epstein “Synthesis of oligomeric anilines” Synthetic Metals 84 119-120 (1997).

The polymeric aniline to be grafted into CNT serves better in conductivity as it increases in molecular weight. However, it decreases in solubility in solvents in proportion to its molecular weight. In addition, when grafted with polymeric aniline of high molecular weight, CNTs are poor in dispersibility. Moreover, high-molecular-weight polymeric aniline has terminal NH, groups poor in reactivity with carboxyl groups on CNTs, which makes grafting difficult. Therefore, the polymeric aniline is preferably composed of 3 to 300 monomers, more preferably 3 to 100 monomers, and further preferably 3 to 32 monomers.

The grafting of polymeric aniline into the CNT having carboxyl groups on its surface can be accomplished by heating both reactants in a solvent in the presence of a condensation agent and a base.

The condensation agent may be freely selected from known ones, such as dicyclohexylcarbodiimide (DCC), diisopropylcarbodiimide (DIC), 1-ethyl-3-(3-dimethylaminopropyl) carbodiimide hydrochloride, and triphenyl phosphite.

The base includes, for example, pyridine and 4-methylaminopyridine without specific restrictions.

Each amount of the condensation agent and the base is 1 to 10 moles for 1 mole of the polymeric aniline.

The solvent for reaction includes, for example, N-methyl-2-pyrrolidone (NMP) and N,N-dimethylformamide (DMF).

The reaction temperature is usually about 20 to 200° C., which is lower than the boiling point of the solvent involved.

The reaction time is usually 12 to 48 hours.

After the reaction is completed, the reaction product is washed with an organic solvent, such as acetone or methanol, capable of dissolving the polymeric aniline, and then filtered out. If necessary, the thus obtained reaction product may be purified by Soxhlet extraction.

The chemically modified carbon nanotubes according to the present invention can be made into a composition by dispersion into an organic solvent of any kind.

The organic solvent for this purpose includes ether compounds such as tetrahydrofuran (THF) and diethyl ether, halogenated hydrocarbons such as methylene chloride and chloroform, amide compounds such as DMF and NMP, ketone compounds such as acetone, methyl ethyl ketone, methyl isobutyl ketone and cyclohexanone, alcohols such as methanol, ethanol, isopropanol and propanol, aliphatic hydrocarbons such as n-heptane, n-hexane and cyclohexane, and aromatic hydrocarbons such as benzene, toluene, xylene and ethylbenzene. Preferable among these solvents is acetone or NMP. These solvents may be used alone or in combination with one another.

The composition according to the present invention may be prepared in any manner by mixing CNT with an organic solvent.

The mixture of CNT and organic solvent should preferably undergo dispersion treatment, such as wet treatment by means of ball mill, beads mill and jet mill, and ultrasonic treatment by means of sonicator of bath type or probe type. Ultrasonic treatment is desirable because of its high efficiency.

Duration of treatment can be 5 minutes to 10 hours, preferably 30 minutes to 5 hours.

The dispersion treatment may be accompanied by heating. The temperature and duration of heating are not specifically restricted. The heating temperature may be near the boiling point of the solvent involved and the duration of heating may be 1 minute to 1 hour, preferably 3 minutes to 30 minutes.

The composition according to the present invention may contain CNT in any concentration low enough for CNT to be dispersed in an organic solvent. An adequate concentration can be about 0.0001 to 10 mass %, preferably about 0.001 to 5 mass %.

The composition according to the present invention may be mixed with a general-purpose synthetic resin or an engineering plastic soluble in the above-mentioned organic solvent.

The general-purpose resin includes, for example, polyolefin resins such as polyethylene (PE), polypropylene (PP), ethylene-vinyl acetate copolymer (EVA) and ethylene-ethyl acrylate copolymer (EEA), styrene resins such as polystyrene (PS), high-impact polystyrene (HIPS), acrylonitrile-styrene copolymer (AS) and acrylonitrile-butadiene-styrene copolymer (ABS), polyvinyl chloride resin, polyurethane resin, phenolic resin, epoxy resin, amino resin, and unsaturated polyester resin.

The engineering plastic includes, for example, polyamide resin, polycarbonate resin, polyphenylene ether resin, modified polyphenylene ether resin, polyethylene terephthalate (PET), polybutylene terephthalate (PBT), polyacetal resin, polysulfone resin, polyphenylenesulfide resin, and polyimide resin.

The CNT-containing composition (solution) according to the present invention can be made into a thin film by coating on a substrate such as PET, glass, and ITO by an adequate method such as casting, spin coating, bar coating, roll coating, and dip coating.

The resulting thin film will find use as a conducting material such as antistatic film and transparent electrode that utilizes the metallic properties of carbon nanotubes, or as a photoelectric conversion element and electroluminescence element that utilize the semiconducting properties of carbon nanotubes.

### EXAMPLES

The present invention will be described below in more detail with reference to Synthesis Examples, Examples, and Comparative Examples, which are not intended to restrict the scope thereof.

### Synthesis Example 1

### Synthesis of MWCNT-COOH

To 400 mL (5.4 mol) of concentrated nitric acid was added 15 g of MWCNT (1 to 25·m long, 10 to 50 nm in diameter, without graphitization), produced by CNT Co., Ltd. After stirring for 24 hours, the treated product was separated by suction filtration. The separated product was added to 400 mL (1 mol) of nitric acid (2.5 mol/L), followed by stirring at 130° C. for 48 hours. The reaction product was separated by suction filtration, thoroughly washed with deionized water, and centrifuged at 3000 rpm. The washed reaction product underwent Soxhlet extraction (with tetrahydrofuran) for 24 hours. Thus, there was obtained 10.5 g of MWCNT-COOH, which is MWCNT having its surface modified with COOH groups (Yield: 70%).

The thus obtained product was identified as the desired product by FT-IR spectroscopy that gave absorption due to the C═O stretching vibration of the carboxyl group at 1710 cm⁻¹ (as shown in FIG. 1). The apparatus for FT-IR is FT-710 made by Horiba Co., Ltd., which has a resolution of 4 and a scanning cycle of 200.

### Synthesis Example 2

### Synthesis of Tetrameric Aniline

In 206 mL (0.021 mol) of 0.1 M HCl aqueous solution was dissolved 2.5 g (0.014 mol) of N-phenyl-1,4-phenylenediamine, and the resulting solution was kept cool at 0° C. In 36 mL (0.004 mol) of 1 M HCl aqueous solution (in a separate container) was dissolved 6.13 g (0.026 mol) of FeCl₃.6H₂O, and the resulting solution was kept cool at 0° C. The two solutions were mixed together by stirring at 0° C. for 4 hours. The reaction product was separated by suction filtration and thoroughly washed with an aqueous solution of 0.1 M HCl. The washed reaction product was added to 150 mL of deionized water, followed by stirring for 2 hours. To the container used for the preceding step was added 1000 mL (0.1 mol) of an aqueous solution of 0.1 M ammonia, followed by stirring for 48 hours. This step is intended for dedoping. The resulting reaction product was separated by suction filtration and then thoroughly washed with an aqueous solution of 0.1 M ammonia. After vacuum drying at 60° C. for 24 hours, there was obtained 1.78 g of tetrameric aniline (4EB) in the form of emeraldine base (Yield: 71%).

The thus obtained product was identified as the desired product by FT-IR spectroscopy that gave absorption due to the benzene ring at 1594 cm⁻¹ and 1504 cm⁻¹ and absorption due to the primary and secondary amines at 3000 to 3500 cm⁻¹ (as shown in FIG. 2). The apparatus for FT-IR is FT-710 made by Horiba Co., Ltd., which has a resolution of 8 and a scanning cycle of 10.

### Example 1

### Grafting of Tetrameric Aniline Onto the Surface of MWCNT

To 100 mL of dehydrated NMP was added 0.4 g (0.16 mmol) of MWCNT-COOH, followed by irradiation with ultrasonic waves for 1 hour under reduced pressure. To the resulting fluid dispersion were sequentially added 0.583 g (1.6 mmol) of 4EB, 1.27 g (16 mmol) of distilled pyridine, and 0.495 g (1.6 mmol) of triphenyl phosphite, followed by stirring at 100° C. for 24 hours. The reaction solution was added to 250 mL of methanol and the reaction product was washed with methanol by suction filtration and finally separated. The thus obtained reaction product was added to 200 mL of methanol. After boiling for 30 minutes, the reaction product was separated by suction filtration and thoroughly washed with methanol. The reaction product was added to 150 mL (0.015 mol) of 0.1 M HCl aqueous solution, followed by stirring for 1 hour, suction filtration, and washing with deionized water. The reaction product was further added to 400 mL (0.04 mol) of aqueous solution of 0.1 M ammonia, followed by stirring for 12 hours, suction filtration, and washing with deionized water. Finally, the reaction product underwent Soxhlet extraction (with acetone) for 10 days. Thus, there was obtained 0.314 g of MWCNT-4EB, which is MWCNT having its surface modified 4EB (Yield: 63%).

The thus obtained product was identified as the desired product by FT-IR spectroscopy that gave absorption due to the benzene nucleus of 4EB at 1562 cm⁻¹ and absorption due to the C═O stretching vibration of the secondary amide at 1675 cm⁻¹, with decreased absorption due to the C═O stretching vibration of the carboxyl group at 1710 cm⁻¹ (as shown in FIG. 3). The apparatus for FT-IR is FT-710 made by Horiba Co., Ltd., which has a resolution of 4 and a scanning cycle of 200.

Incidentally, elemental analysis suggests that the amount of 4EB for MWCNT is 22.7 mass %.

### Example 2

### Liquid Dispersion of MWCNT-4EB In NMP

To NMP was added the MWCNT-4EB synthesized in Example 1 such that the amount of MWCNT was 0.1 mass %, followed by irradiation with ultrasonic waves (30 W) for 1 hour.

Upon observation under a polarization microscope (BX50 made by Olympus Corporation), the resulting liquid dispersion was found to contain MWCNT uniformly dispersed in the solvent. Good dispersion remained without precipitation of MWCNT after standing at room temperature for 2 months.

### Example 3

### Liquid Dispersion of MWCNT-4EB In Acetone

The same procedure as in Example 2 was repeated to prepare a liquid dispersion of MWCNT-4EB except that NMP was replaced by acetone.

Upon observation under a polarization microscope, the resulting liquid dispersion was found to contain MWCNT uniformly dispersed in the solvent. Good dispersion remained without precipitation of MWCNT after standing at room temperature for 2 months.

### Example 4

### Liquid Dispersion of MWCNT-4EB In NMP

The same procedure as in Example 2 was repeated to prepare a liquid dispersion of MWCNT-4EB except that the amount of MWCNT was changed to 0.3 mass %.

Upon observation under a polarization microscope, the resulting liquid dispersion was found to contain MWCNT uniformly dispersed in the solvent. Good dispersion remained without precipitation of MWCNT after standing at room temperature for 2 months.

### Example 5

### Liquid Dispersion of MWCNT-4EB In Acetone

The same procedure as in Example 3 was repeated to prepare a liquid dispersion of MWCNT-4EB except that the amount of MWCNT was changed to 0.3 mass %.

Upon observation under a polarization microscope, the resulting liquid dispersion was found to contain MWCNT uniformly dispersed in the solvent. Good dispersion remained without precipitation of MWCNT after standing at room temperature for 2 months.

### Example 6

### Liquid Dispersion of MWCNT-4EB In NMP

The same procedure as in Example 2 was repeated to prepare a liquid dispersion of MWCNT-4EB except that the amount of MWCNT was changed to 0.5 mass %.

Upon observation under a polarization microscope, the resulting liquid dispersion was found to contain MWCNT uniformly dispersed in the solvent. Good dispersion remained without precipitation of MWCNT after standing at room temperature for 2 months.

### Example 7

### Liquid Dispersion of MWCNT-4EB In Acetone

The same procedure as in Example 3 was repeated to prepare a liquid dispersion of MWCNT-4EB except that the amount of MWCNT was changed to 0.5 mass %.

Upon observation under a polarization microscope, the resulting liquid dispersion was found to contain MWCNT uniformly dispersed in the solvent. Good dispersion remained without precipitation of MWCNT after standing at room temperature for 2 months.

Each of the liquid dispersions obtained in Examples 2, 4, and 6 mentioned above was applied to a glass substrate by doctor blade coating, bar coating, or spin coating, whose apparatuses are variable doctor blade made by Tester Sangyo, automatic coater PI-1210 made by Tester Sangyo, and SPINCOATER 1H-D7 made by MIKASA, respectively. The resulting thin film was observed under a scanning electron microscope. It was found that MWCNT formed a network structure on the substrate. The result of Example 2 is shown in FIG. 7.

The film thickness was controlled by adjusting the coating rate in the case of doctor blade coating or by adjusting the concentration of MWCNT in the liquid dispersion in the case of spin coating. (The doctor blade was set at 7, which is the top of the range from 1 to 7.)

The results are shown in Table 1.

TABLE 1

| | | | | | Dispers- |
|---|---|---|---|---|---|
| | Concentration | Film | | Dispers- | ibility |
| | of MWCNT | forming | Coating | ibility | on glass |
| | (mass %) | method | rate | in solvent | substrate |
| Example | 0.1 | Spin | — | ∘ | ∘ |
| 2 | | coating | | | |
| Example | 0.3 | Spin | — | ∘ | ∘ |
| 4 | | coating | | | |
| Example | 0.5 | Doctor | 3 | ∘ | ∘ |
| 6 | | blade | | | |
| | | Doctor | 5 | ∘ | ∘ |
| | | blade | | | |
| | | Doctor | 7 | ∘ | ∘ |
| | | blade | | | |
| | | Spin | — | ∘ | ∘ |
| | | coating | | | |

Remarks:
(1) Dispersibility in solvent
∘: There exist no visible aggregates (smaller than several micrometers).
x: There exist visible aggregates (larger than tens of micrometers).
(2) Dispersibility on glass substrate
∘: There exist no visible aggregates (smaller than several micrometers).
x: There exist visible aggregates (larger than tens of micrometers).

### Comparative Example 1

### Liquid Dispersion of MWCNT In NMP

To NMP was added MWCNT (0.1 mass %), followed by irradiation with ultrasonic waves (30 W) for 1 hour.

Upon observation under a polarization microscope, the resulting liquid dispersion was found to contain large aggregates of MWCNT in the solvent. MWCNT precipitated after standing at room temperature for 2 months.

### Comparative Example 2

### Liquid Dispersion of MWCNT In Acetone

The same procedure as in Comparative Example 1 was repeated except that NMP was replaced by acetone.

Upon observation under a polarization microscope, the resulting liquid dispersion was found to contain large aggregates of MWCNT in the solvent. MWCNT precipitated after standing at room temperature for 2 months.

### Comparative Example 3

### Liquid Dispersion of MWCNT-COOH In NMP

To NMP was added MWCNT-COOH such that the amount of MWCNT is 0.1 mass %, followed by irradiation with ultrasonic waves (30 W) for 1 hour.

Upon observation under a polarization microscope, the resulting liquid dispersion was found to contain large aggregates of MWCNT in the solvent. MWCNT precipitated after standing at room temperature for 2 months.

The liquid dispersion was applied to a glass substrate by spin coating and the resulting thin film was observed under a scanning electron microscope. It was found that MWCNT forms aggregates on the substrate (as shown in FIG. 8).

### Comparative Example 4

### Liquid Dispersion of MWCNT-COOH In Acetone

The same procedure as in Comparative Example 3 was repeated except that NMP was replaced by acetone.

Upon observation under a polarization microscope, the resulting liquid dispersion was found to contain large aggregates of MWCNT in the solvent. MWCNT precipitated after standing at room temperature for 2 months.

The liquid dispersion was applied to a glass substrate by spin coating and the resulting thin film was observed under a scanning electron microscope. It was found that MWCNT forms aggregates on the substrate.

### Comparative Example 5

### Liquid Dispersion of MWCNT-COOH In NMP

The same procedure as in Comparative Example 3 was repeated except that MWCNT was added such that the amount of NMP was 0.5 mass %.

Upon observation under a polarization microscope, the resulting liquid dispersion was found to contain large aggregates of MWCNT in the solvent (as shown in FIG. 5). MWCNT precipitated after standing at room temperature for 2 months.

The liquid dispersion was applied to a glass substrate by spin coating and the resulting thin film was observed under a scanning electron microscope. It was found that MWCNT forms aggregates on the substrate.

### Comparative Example 6

### Liquid Dispersion of MWCNT-COOH In Acetone

The same procedure as in Comparative Example 4 was repeated except that MWCNT-COOH was added such that the amount of NMP was 0.5 mass %.

Upon observation under a polarization microscope, the resulting liquid dispersion was found to contain large aggregates of MWCNT in the solvent. MWCNT precipitated after standing at room temperature for 2 months.

The liquid dispersion was applied to a glass substrate by spin coating and the resulting thin film was observed under a scanning electron microscope. It was found that MWCNT forms aggregates on the substrate.

### Comparative Example 7

### Liquid Dispersion of MWCNT-COOH+4EB Post Blend In NMP

To NMP was added MWCNT-COOH such that the amount of MWCNT was 0.5 mass % and further added 4EB in the same amount as 4EB in MWCNT-4EB (22.7 mass % for MWCNT), followed by irradiation with ultrasonic waves (30 W) for 1 hour. The resulting liquid dispersion was found to contain a large number of aggregates (as shown in FIG. 6).

The results of Comparative Examples 1, 3, and 7 are shown in Table 2.

TABLE 2

| | | | | Dispers- |
|---|---|---|---|---|
| | Concentration | | Dispers- | ibility |
| | of MWCNT | Film forming | ibility | on glass |
| | (mass %) | method | in solvent | substrate |
| Comparative | 0.1 | — | x | — |
| Example | | | | |
| 1 | | | | |
| Comparative | 0.1 | Spin coating | x | x |
| Example | | | | |
| 3 | | | | |
| Comparative | 0.5 | — | x | — |
| Example | | | | |
| 7 | | | | |

### BRIEF DESCRIPTION OF THE DRAWINGS

FIG. 1 is a diagram showing the FT-IR spectrum of MWCNT-COOH obtained in Synthesis Example 1;

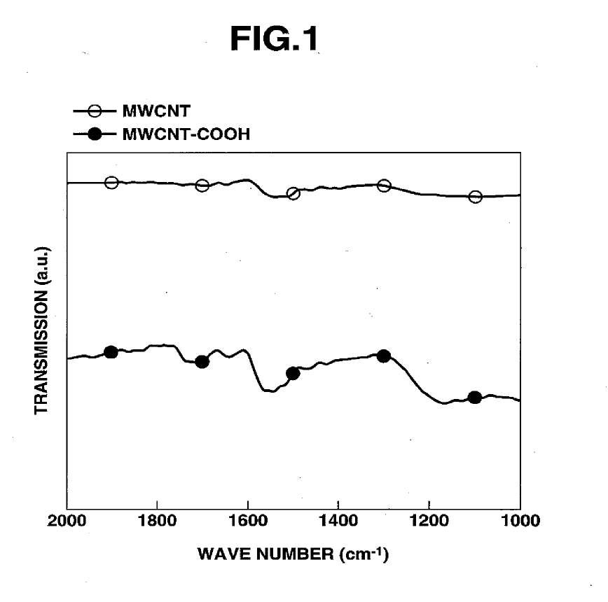

FIG. 2 is a diagram showing the FT-IR spectrum of tetrameric aniline (4EB) obtained in Synthesis Example 2;

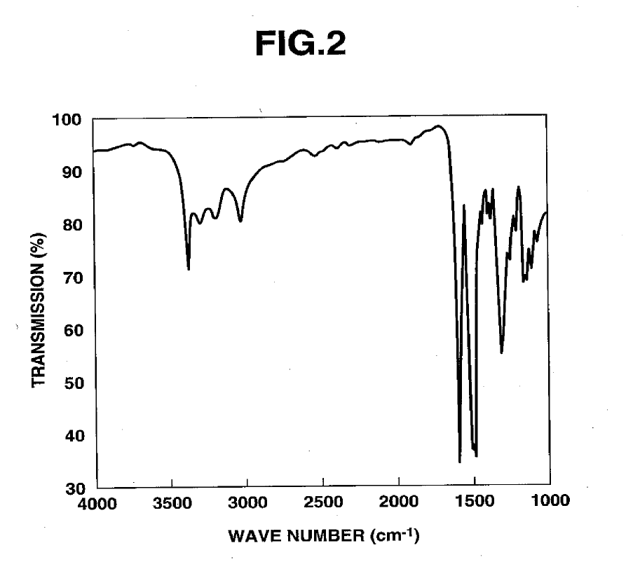

FIG. 3 is a diagram showing the FT-IR spectrum of MWCNT-4EB obtained in Example 1;

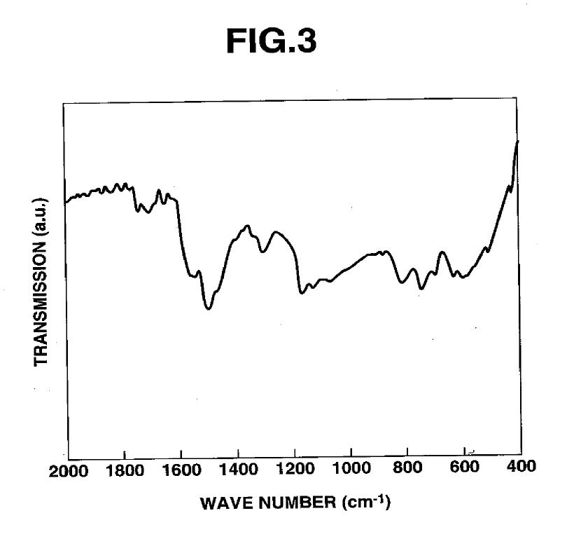

FIG. 4 is a photograph showing a liquid dispersion (0.5 mass %) of MWCNT-4EB (prepared in Example 6) sticking to the wall of a container;

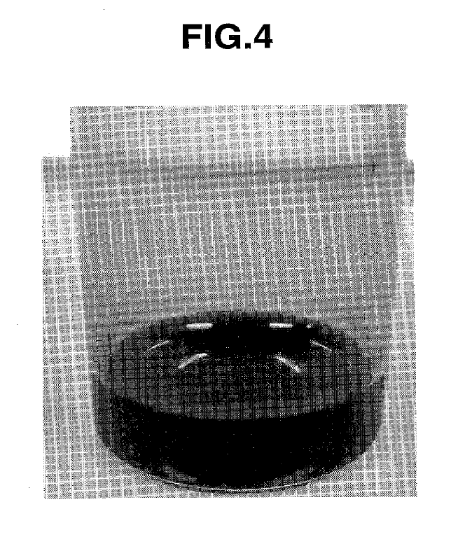

FIG. 5 is a photograph showing a liquid dispersion (0.5 mass %) of MWCNT-COOH (prepared in Comparative Example 5) sticking to the wall of a container;

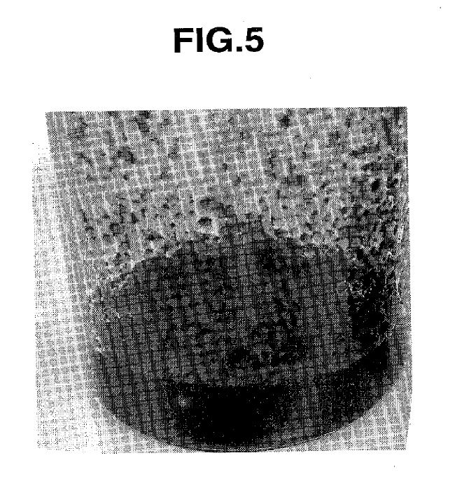

FIG. 6 is a photograph showing a post-blend liquid dispersion (0.5 mass %) of MWCNT-COOH+4EB (prepared in Comparative Example 7) sticking to the wall of a container;

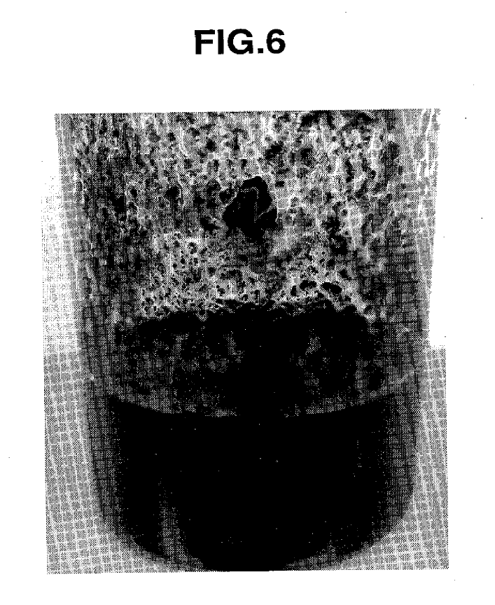

FIG. 7 is a scanning electron microscope (SEM) photograph of a thin film formed from a liquid dispersion (0.1 mass %) of MWCNT-4EB prepared in Example 2; and

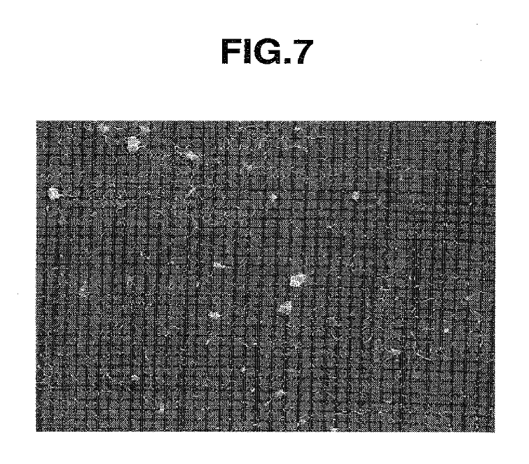

FIG. 8 is a SEM photograph of a thin film formed from a liquid dispersion (0.1 mass %) of MWCNT-COOH prepared in Comparative Example 3.

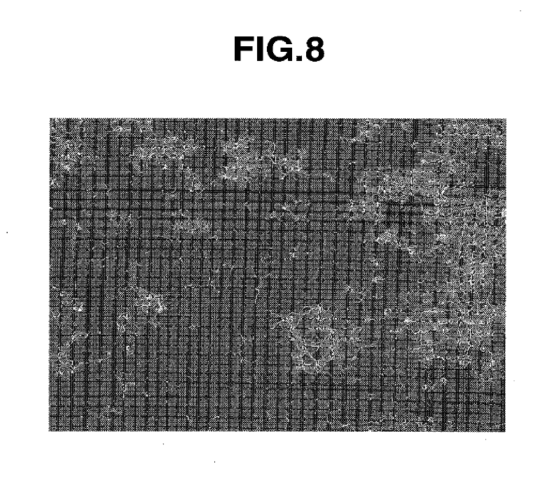

1. A method for producing chemically modified carbon nanotubes which comprises:
heating carbon nanotubes having carboxyl groups on the surface thereof and polymeric aniline in a solvent in the presence of a condensation agent and a base to graft said polymeric aniline to said carbon nanotubes to produce said modified carbon nanotubes having said polymeric aniline bound to said nanotubes through an amide linkage, so that chemically modified carbon nanotubes having an unsubstituted phenyl group at the end of a polyaniline chain are obtained.

2. The method according to claim 1, wherein said carbon nanotubes are multiwall carbon nanotubes.

3. The method according to claim 1, wherein said carbon nanotubes contain said carboxyl groups in an amount of 0.1 to 1 mmol/g.

4. The method according to claim 1, wherein said polymeric aniline is 3- to 300-meric aniline.
